(19) United States
(12) Patent Application Publication
(10) Pub. No.: US 2024/0260970 A1
(43) Pub. Date: Aug. 8, 2024

(54) SYSTEM, APPARATUS AND METHOD COMPRISING A SMART DRILL

(71) Applicant: Alexander M. Crespo, Chicago, IL (US)

(72) Inventor: Alexander M. Crespo, Chicago, IL (US)

(73) Assignee: Alexander M. Crespo, Chicago, IL (US)

(21) Appl. No.: 18/433,324

(22) Filed: Feb. 5, 2024

Related U.S. Application Data
(60) Provisional application No. 63/483,126, filed on Feb. 3, 2023.

(51) Int. Cl. A61B 17/16; A61B 17/00; A61B 90/00

(52) U.S. Cl. CPC A61B 17/1615; A61B 17/1626; A61B 17/1668; A61B 90/06; A61B 2017/00022; A61B 2090/061; A61B 2090/067

(57) ABSTRACT

A system, apparatus and method comprising a smart drill is provided. The smart drill comprises a first end and a second end located at a distal end of the first end; a threaded portion in proximity to the second end; and a shaft connected to the threaded portion. The smart drill is configured with at least one embedded processor configured to execute computer executable instructions and configured to provide real-time intra-operative reading of at least one variable measurement parameter of at least one bone during a certain medical procedure. The variable measurement parameter can be a length, an alignment or a rotation. The system can also measure all three of length, alignment and rotation. The smart drill bit comprising the processor is further configured to connect with at least one sensor in order to sense a three-dimensional positioning or position in space. The processor is further configured to connect and communicate with at least one external device over at least one network.

### CROSS REFERENCE TO OTHER APPLICATION

This application claims the benefit of and priority to U.S. Provisional Application No. 63/483,126 filed Feb. 3, 2023, the contents of which are incorporated herein by reference.

### FIELD OF THE DISCLOSURE

The present disclosure generally relates to a system, apparatus and method of using a drill bit configured to allow real-time measurements (e.g., intra-operative measurement) of various parameters related to medical procedures involving certain kinds of fractures. More specifically, the present disclosure comprises a smart drill configured to allow the real-time intra-operative measurement of certain variables (for example, length, alignment and rotation of a long bone) during certain medical procedures for certain kinds of fractures.

### BACKGROUND

As is well known in the field of medical science, the skeletal system of a human includes many bones including long bones such as the femur, fibula, tibia, humerus, radius and ulna. These long bones are particularly exposed to trauma from accidents and as such are often fractured during such trauma and may result in disability. For bone injuries, especially those resulting from one or more fractures of the long bone, one or more fixation devices can be used to fix the fracture pieces and stabilize the long bone. In other words, fixation tools and devices, which are available in a wide variety of different shapes and sizes, have long been used in the repair of bone defects, including long bone fractures.

During certain medical procedures, an operator typically sets the bone to be repaired in the proper position and then uses the fixation tools and devices to secure the bone in that position for healing. A fixation device, such as a bone plate or rod, can be secured to the bone by a fixation tool, such as a bone screw. Alternatively, a bone screw or other similar device can be used by itself to repair a bone defect. In addition, certain uses of drill bits in some medical procedures are well known. Further, conventional prior art has provided some options for using non-smart drill bits for certain kinds of medical operations, even if they lack in configurability and smart connectivity that of the smart drill bit disclosed herein.

Finally, in an attempt to improve the options for available drill bits, prior art provides various options for drill bits. For instance, a multi-functional orthopedics perforator drill which can directly handle the fractured bone segments and can provide benefits such as reduction in the cost of operation and shortening of the operating time for the specific medical procedure. However, as further disclosed herein, none of these conventional apparatus or methods come close to the novel and innovative smart drill disclosed in this application.

It is further known within the medical industry, especially in the field of orthopedics, that there is a risk of damage to adjacent body parts including muscles, tendons, skin, organs and the like during certain medical procedures. To alleviate such concerns, conventional and prior art apparatus and methods have tried various options; for example, installation of certain fixation devices into the long bone segment that serve as guides for drilling, reaming, screw-tapping, placement of the guide wire and/or using certain procedures for confirming the site of the drilled bore hole with x-rays, prior to drilling, attempting to provide stability and guidance during drilling or the insertion of devices, such as bone screws, into the bone. Even though these apparatus and methods offer certain advantages, they also have various shortcomings and disadvantages that the disclosed invention addresses in a very efficient way.

It is to be noted that with certain bone injuries/fractures, especially those resulting from one or more fractures of the long bone, one or more fixation devices can be used to fix the fractured pieces and stabilize the long bone. Fractures can be treated with screws or other fixation devices to stabilize the bone, which are inserted into or through the bone once the fracture site has been guided to the correct alignment. For instance, it is known that bone parts or fragments involved in fractures of the femur are difficult to stabilize satisfactorily. More particularly, as is well known in the field of medical science, for operations related to long bone fractures there is a need to provide orthopedic surgeons with real-time, intra-operative measurements of parameters regarding the length, alignment and rotation of a fractured long bone (e.g., femur, tibia, etc.).

Current conventional and prior art apparatus, systems and methods use various processes such as intra-operative fluoroscopy (x-ray) in order to measure the length, alignment and rotation of a broken long-bone. However, conventional and prior art apparatus and systems—e.g., intra-operative fluoroscopy—are exceedingly difficult to be accomplished in an accurate and precise fashion, as has been proven in multiple studies in the field of medical science. For instance, there is a conventional prior art technology referred to as the “navigation” technology in orthopedics that primarily uses sensors and robotic arms to detect position in space. However, as noted above such conventional prior art apparatus and methods have shortcomings and are inefficient and do not provide the advantage of the innovative smart drill disclosed herein.

There are currently two key disadvantages to the different types of methods currently used or under consideration within the medical industry. The first one is known as the “fluoroscopy method”—a method in which it is (1) difficult and challenging to image an entire bone on one film; (2) exceptionally difficult to accurately measure (especially rotation); and (3) takes an inordinate amount of time, which is generally not in abundance due to the nature of the procedure, in order to provide all the images and measurements needed by the medical professional to make an informed judgment during the operative procedure.

The second key disadvantage is the use of the “navigation method,” which doesn't exist yet in a practical sense, the “navigation method” requiring a probe to be set up as well as procuring a robot to sense three-dimensional positioning of the probes, which can cost an upwards of $1M of investment, at a minimum. In addition, the “navigation method,” using a robot to sense three-dimensional positioning of the probes, requires bulky equipment which is generally not familiar to or preferred by orthopedic trauma surgeons. Thus, the current designs related to the “navigation method” or navigation robots provides—at best—a cumbersome and inefficient combination of a rigid robotic arm and navigation capabilities for measuring certain alignments in spinal surgery.

In sum, conventional devices do not have the smart technology that provides the orthopedic trauma surgeon with real-time feedback regarding restoration of length, alignment and rotation of a fractured long bone—a “smart drill bit” that allows for real-time intra-operative measurement of length, alignment and rotation of a long bone.

In view of the foregoing disadvantages of prior art systems in the relevant field of medical/orthopedics, there is clearly a market need for an improved and smart drill that will be able to overcome the noted disadvantages in the currently available apparatus, methods and systems.

Therefore, there is a need for an improved apparatus, method and system comprising a novel smart drill—providing a simple and efficient means of overcoming the prior art shortcomings and disadvantages of currently available conventional equipment-configured to provide real-time intra-operative measurement of various parameters required for medical procedures related to certain kinds of fractures.

More specifically, the present disclosure comprises a smart drill configured to allow the real-time intra-operative measurement of length, alignment and rotation of a long bone related to certain kinds of fractures. The present invention is directed to an improved smart drill to provide the orthopedic surgeon or other medical professional with real-time, intra-operative measurements (for example, length, alignment and rotation of a fractured long bone such as femur, tibia, etc.). The novel smart drill disclosed herein provides significant technological advantages from conventional apparatus, methods and systems while overcoming the disadvantages of such conventional/prior art systems, apparatus, methods as further discussed below.

### SUMMARY

Embodiments disclosed in the present disclosure provide a system, apparatus, methods comprising a novel and unique smart drill configured to provide the real-time intra-operative measurement of certain parameters of a long bone during certain medical procedures for certain kinds of fractures.

A smart drill comprises a first end and a second end located at a distal end of the first end; a threaded portion in proximity to the second end; and a shaft connected to the threaded portion. The smart drill is configured with at least one embedded processor configured to execute computer executable instructions and configured to provide real-time intra-operative reading of at least one variable measurement parameter of at least one bone during a certain medical procedure. The variable measurement parameter can be a length, an alignment or a rotation. The system can also measure all three of length, alignment and rotation. The smart drill bit comprising the processor is further configured to connect with at least one sensor (which could be an embedded sensor or a sensor in communication with the smart device/processor within the smart drill) in order to sense a three-dimensional positioning in space. The processor is further configured to connect and communicate with at least one external device over at least one network.

In an aspect of the present disclosure, a smart drill system comprises at least one drill bit having a first end and a second end located at a distal end of the first end; a threaded portion in proximity to the second end; a shaft connected to the threaded portion; wherein the at least one drill bit is configured with at least one embedded processor configured to execute computer executable instructions, wherein the system is further configured to: to provide real-time intra-operative reading of at least one variable measurement parameter of at least one bone during a certain medical procedure.

In another aspect of the present disclosure, a method of using a smart drill (or a smart drill bit) comprises the steps of: (1) configuring at least one drill bit having a first end and a second end located at a distal end of the first end, a threaded portion in proximity to the second end, and a shaft connected to the threaded portion; (2) further configuring the at least one drill bit with at least one embedded processor in order to execute computer executable instructions; and (3) providing real-time intra-operative reading of at least one variable measurement parameter of at least one bone during a certain medical procedure.

In yet another aspect of the present disclosure, an improved smart drill embedded with smart drill technology provides the orthopedic trauma surgeon or other similar medical professional with real-time feedback regarding restoration of length, alignment and rotation of a fractured long bone. In the depicted embodiment, by way of example and not of limitation, the smart drill bit can be configured with a smart device (e.g., wireless chip/embedded processor) wherein the smart device is impregnated within the shaft and configured to connect with external devices. These external devices can be within the same or different networks for connectivity to the smart device embedded within the smart drill, as disclosed herein.

In still yet another aspect of the present disclosure, the apparatus, method and system comprises of a plurality of smart drills (or smart drill bits) having a plurality of embedded smart devices (e.g., wireless chip/embedded processor) and configured with the ability to sense the three-dimensional positioning in space and provide measurement of length, alignment and rotation (i.e., functioning as a virtual ruler and protractor within the device). In the depicted embodiment, by way of example and not of limitation, the plurality of smart drills comprises at least two smart drill bits configured with at least two smart devices (e.g., wireless chips/embedded processors) wherein each smart device is impregnated/embedded within the shaft and configured to connect with external devices. It is to be noted that the external devices can be within the same or different networks for connectivity to the at least two smart devices embedded within the at least two smart drill bits.

In another aspect of the present disclosure, the smart drill bits comprising smart devices are further configured with sensors (e.g., wireless sensors or other types of sensors providing equivalent functionality of connectivity) in order to communicate via Bluetooth or other short-range wireless technology standard that can be used for exchanging data between the smart drill and other devices (e.g., mobile devices, handheld devices such as iPad or other tablets/personal computers connected via network) over certain distances or communicate via Bluetooth or other short-range wireless technology standard within an “app” that can provide a clear and accurate readout of the measurement values to a medical professional.

In yet another aspect of the present disclosure, the ability to engineer and design a smart drill with a wireless chip impregnated/embedded within the shaft is provided (also illustrated in the accompanying FIGS. 1-5).

In the depicted embodiment, the ability for at least two smart drill bits with at least two embedded smart chips within the drill bits is provided in order to sense their three-dimensional position in space and accurately measure length, alignment and rotation (i.e., function as a virtual ruler and protractor) (illustrated in FIGS. 2-4); and the ability for the wireless sensor to communicate via Bluetooth or other short-range wireless technology standard that can be used for exchanging data between the smart drill and other devices (e.g., mobile devices, handheld devices such as iPad or other tablets/personal computers connected via network) over certain distances or communicate via Bluetooth or other short-range wireless technology standard within an “app” that can provide a clear and accurate readout of the measurement values to a medical professional is provided (illustrated in FIG. 5). In the depicted embodiment, the apparatus, method and system comprises a plurality of a smart drills (or smart drill bits) having a plurality of embedded smart devices (e.g., wireless chips/embedded processors) and configured with the ability to sense the three-dimensional positioning in space and provide measurement of length, alignment and rotation (i.e., functioning as a virtual ruler and protractor within the device). By way of example and not of limitation, the plurality of a smart drill comprises at least two smart drill bits configured with at least two smart devices (e.g., wireless chips/embedded processors) wherein each of the smart devices is impregnated/embedded within the shaft and configured to connect with external devices. It is to be noted that the external devices can be within the same or different networks for connectivity to the at least two smart devices embedded within the at least two smart drill bits.

This summary is provided to introduce a selection of concepts in a simplified form that are further described in the detailed description of the disclosure. This summary is not intended to identify key or essential inventive concepts of the claimed subject matter, nor is it intended for determining the scope of the claimed subject matter. The references made above in detail to the embodiments of the disclosure are provided by way of explanation of the disclosure, not in limitation of the disclosure. It will be apparent to those skilled in the art that various modifications and variations can be made in the present disclosure without departing from the scope or spirit of the disclosure.

Features illustrated or described as part of one embodiment can be used on another embodiment to yield a still further embodiment. Thus, it is intended that the present disclosure cover such modifications and variations as come within the scope of the appended claims and their equivalents. Other objects, features and aspects of the present disclosure are disclosed in the following detailed description. It is to be understood by one of ordinary skill in the art that the present discussion is a description of exemplary embodiments only and is not intended as limiting the broader aspects of the present disclosure, which broader aspects are embodied in the exemplary constructions.

### BRIEF DESCRIPTION OF THE DRAWINGS

The disclosure will be more readily understood in view of the following description when accompanied by the below figures. The accompanying figures incorporated in and forming a part of the specification illustrate several aspects of the present invention and, together with the description, serve to explain the principles of the invention.

To make the objects, technical solutions and advantages of the present invention clearer, the drawings in the present invention will be combined to clearly and completely describe the technical solutions of the present invention, and obviously, the described embodiments are some, but not all, embodiments of the present invention. Based on the embodiments in the present invention, all other embodiments obtained by a person skilled in the art related to the invention disclosed herein belong to the protection scope of the present invention.

In the description of the present invention, it is to be noted that, unless otherwise explicitly specified or limited, the terms “mounted” and “connected” are to be construed broadly, and may be, for example, fixedly connected, detachably connected or integrally connected; can be mechanically or electrically connected; may be connected directly or indirectly through intervening media; or may be interconnected between two elements. The specific meaning of the above terms in the present invention can be understood in specific cases to those skilled in the art.

FIG. 1 illustrates a smart drill bit comprising a smart device according to one or more aspects of the present disclosure.

FIG. 2 illustrates the demonstration of length via a smart drill bit according to one or more aspects of the present disclosure.

FIG. 3 illustrates the demonstration of alignment in the coronal plane within the fracture site portending a certain angle according to one or more aspects of the present disclosure.

FIG. 4 illustrates the demonstration of rotation in the axial plane (as shown, Bit 1 is placed in the femoral head/neck segment and Bit 2 is placed transversely across the distal femur) portending a certain angle according to one or more aspects of the present disclosure.

FIG. 5 illustrates the real-time intra-operative readout provided on an iPad or “app” that provides the surgeon with immediate feedback of length, alignment and rotation according to one or more aspects of the present disclosure.

### DETAILED DESCRIPTION OF THE DISCLOSURE

For the purposes of promoting and understanding the principles disclosed herein, reference is now made to the preferred embodiments illustrated in the drawings, and specific language is used to describe the same. It is understood that no limitation of the scope of the disclosure is hereby intended. Such alterations and further modifications in the illustrated apparatus and such further applications of the principles disclosed and illustrated herein are contemplated as would normally occur to one of ordinary skill in the art to which this disclosure relates.

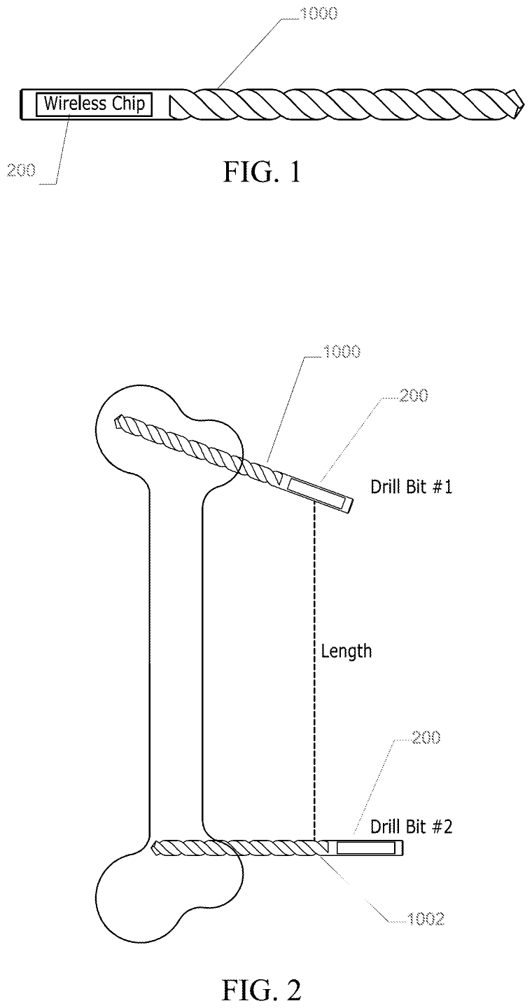

As illustrated in FIGS. 1-4, a smart drill (1000) comprises a first end and a second end located at a distal end of the first end; a threaded portion in proximity to the second end; and a shaft connected to the threaded portion. The smart drill 1000 is configured with at least one embedded processor (e.g., a wireless chip 200) configured to execute computer executable instructions and configured to provide real-time intra-operative reading of at least one variable measurement parameter of at least one bone during a certain medical procedure.

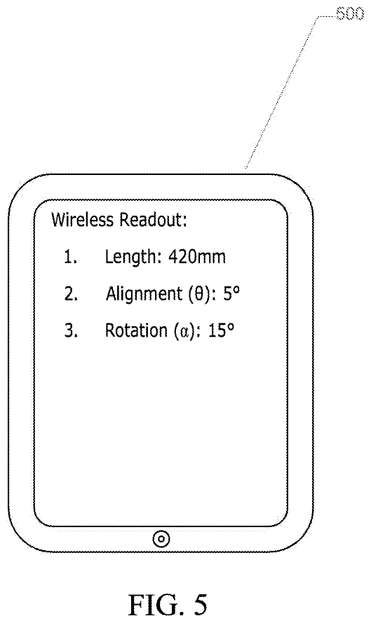

The improved smart drill 1000 embedded with a processor 200 (smart drill technology) disclosed herein provides the orthopedic trauma surgeon or other similar medical professional with real-time feedback regarding restoration of length, alignment and rotation of a fractured long bone. In the depicted embodiment, by way of example and not of limitation, the smart drill bit 1000 can be configured to connect over a network with an impregnated smart device 200 (e.g., wireless chip/embedded processor) the smart device impregnated within the shaft and configured to connect with at least one external device (see FIG. 5, 500). The at least one external device (500) can be within (or in) the same or one or more different networks for connectivity to the smart device 200 embedded within the smart drill 1000, as disclosed herein.

FIG. 1 also illustrates a smart drill bit 1000 comprising a smart device 200 (e.g., a wireless chip) inside the shaft of the drill bit 1000 that allows the smart drill (or smart drill bit) 1000 to sense its position in space. As disclosed herein and further illustrated in FIGS. 1-4, the apparatus, methods and system comprises a plurality of drill bits or smart drill bits—at least two drill bits (1000, 1002) placed in certain pre-configured and/or pre-determined/appropriate positions within the bone such that the two drill bits (1000, 1002) can be configured to measure variables such as length, alignment and rotation of the long bone. The smart devices (200) embedded within the shaft of the drill can then be further configured to measure the distance between Bit 1 (1000) and Bit 2 (1002) (length), angle portended by Bit 1 (1000) and Bit 2 (1002) in the coronal plane (alignment), and angle portended by Bit 1 (1000) and Bit 2 (1002) in the axial plane (rotation). FIG. 2 illustrates the demonstration of length via a smart drill bit according to one or more aspects of the present disclosure.

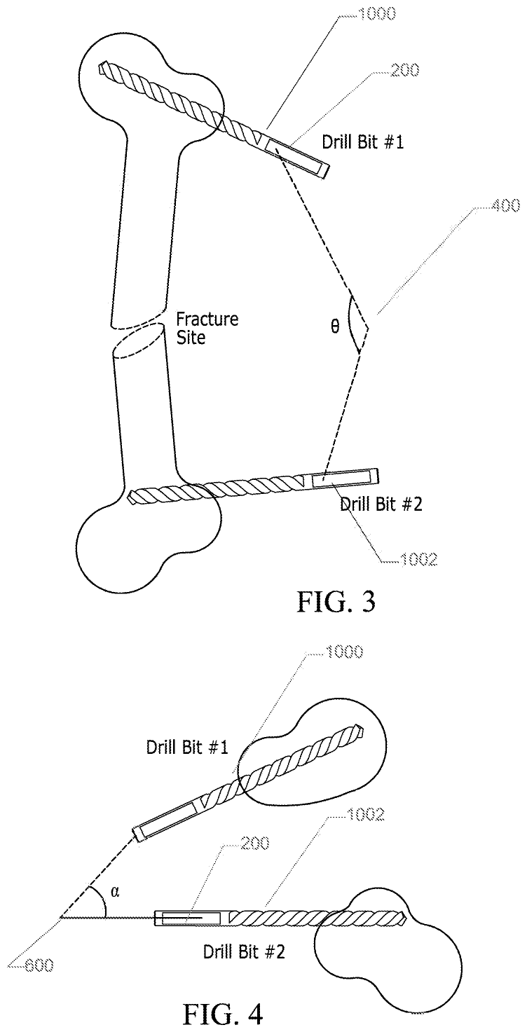

As further illustrated, FIG. 1 an improved drill bit 1000 comprising a wireless chip inside the shaft of the drill bit 1000 allows the bit 1000 to sense its position in space. The wireless chip 200 is shown as an example. FIG. 3 illustrates the demonstration of alignment in the coronal plane within the fracture site portending a certain angle according to one or more aspects of the present disclosure. FIG. 4 illustrates the demonstration of rotation in the axial plane (as shown, Bit 1 is placed in the femoral head/neck segment and Bit 2 is placed transversely across the distal femur) portending a certain angle according to one or more aspects of the present disclosure. FIG. 5 illustrates the real-time intra-operative readout provided on an iPad or “app” that provides the surgeon with immediate feedback of length, alignment and rotation according to one or more aspects of the present disclosure.

The apparatus, method and system illustrated in FIGS. 2 and 3 shows at least two drill bits (1000, 1002) placed in a certain pre-configured/pre-determined (and appropriate) locations/positions within the bone so that it can measure length, alignment and rotation of the long bone. The wireless chips 200 or other kind of smart chips will then be able to measure the distance between the drill bits (e.g., Bit 1, 1000 and Bit 2, 1002 (length), angle portended by Bit 1 and Bit 2 in the coronal plane (alignment), and angle portended by Bit 1 and Bit 2 in the axial plane (rotation)). FIG. 2 also illustrates the demonstration of length for the smart drill bit disclosed and claimed herein. FIG. 3 illustrates the alignment in the coronal plane with fracture site of a bone and Bit 1 and Bit 2 illustrate and portend a certain angle.

FIG. 4 illustrates the rotation in the axial plane. Bit 1 is placed in the femoral head/neck segment, and Bit 2 is placed transversely across the distal femur. Bit 1 and Bit 2 portend angle Alpha.

FIG. 5 depicts the real-time intra-operative readout provided, by way of example and not of limitation, on a smart device (e.g., iPad) or “app” that can provide the surgeon with immediate feedback of length, alignment and rotation via usage of the smart drill bit.

In another aspect of the present disclosure, the apparatus, method and system comprises of a plurality of smart drill bits (1000, 1002) having a plurality of embedded smart devices 200 (e.g., wireless chips/embedded processors) and configured with the ability to sense the three-dimensional positioning in space and provide measurement of length, alignment and rotation (i.e., functioning as a virtual ruler and protractor within the device). In the depicted embodiment, by way of example and not of limitation, the plurality of smart drills comprises at least two smart drill bits (1000, 1002), each configured with at least one smart device 200 (e.g., wireless chips/embedded processors) wherein each smart device 200 is impregnated/embedded within the shaft of the drill (1000, 1002) and configured to connect with at least one external device (500).

It is to be noted that the external device can be within the same or in one or more different networks for connectivity to the at least two smart devices (200) embedded within the two smart drill bits (1000, 1002). An exemplary embodiment is shown in FIG. 1, comprising the two drill bits to be placed in certain configuration/position within the bone so that it can measure length, alignment and rotation of the long bone. The smart devices embedded within the shaft of the drill can then be configured to measure the distance between Bit 1 and Bit 2 (length), angle portended by Bit 1 and Bit 2 in the coronal plane (alignment), and angle portended by Bit 1 and Bit 2 in the axial plane (rotation).

In yet another aspect of the present disclosure, the smart drill bits (1000, 1002), each having at least one smart device (200) can be further configured with sensors (e.g., wireless sensors or other types of sensors providing equivalent functionality of connectivity) in order to communicate via Bluetooth or other short-range wireless technology standard that can be used for exchanging data between the smart drill (1000, 1002) and other devices (e.g., mobile devices, handheld devices such as iPad or other tablets/personal computers connected via network, 500) over certain distances or communicate via Bluetooth or other short-range wireless technology standard within an “app” that can provide a clear and accurate readout of the measurement values to a medical professional.

In yet another aspect of the present disclosure, the ability to engineer, design and manufacture a smart drill 1000 with a wireless chip 200 impregnated/embedded within the shaft is provided (also illustrated in FIG. 1). As further illustrated in the accompanying figures (FIGS. 1-5), the at least two smart drill bits (1000, 1002) with at least two embedded smart chips (one embedded smart chip 200 in each drill bit) is configured to provide coordinates (or other similarly measured variables) in order to measure and sense their three-dimensional position in space and accurately measure length, alignment and rotation (i.e., function as a virtual ruler and protractor) as illustrated in FIGS. 2-4). Further, the wireless sensor (not shown in the drawings) has the ability to communicate via Bluetooth or other short-range wireless technology standard that can be used for exchanging data between the smart drill (1000, 1002) and other devices (e.g., mobile devices, handheld devices such as iPad or other tablets/personal computers connected via network, as shown in FIG. 5) over certain distances or communicate via Bluetooth or other short-range wireless technology standard within an “app” that can provide a clear and accurate readout of the measurement values to a medical professional is provided (illustrated in FIG. 5).

The apparatus, method and system comprising a plurality of smart drills (or smart drill bits 1000, 1002) having a plurality of embedded smart devices 200 (e.g., wireless chips/embedded processors) is further configured with the ability to sense the three-dimensional positioning in space and provide measurement of length, alignment and rotation (i.e., functioning as a virtual ruler and protractor within the device). By way of example and not of limitation, the plurality of smart drills comprises at least two smart drill bits configured with at least smart devices (e.g., wireless chips/embedded processors) wherein each of the smart devices is impregnated/embedded within the shaft and configured to connect with a plurality of external devices. It is to be noted that the plurality of external devices can be within the same network or different networks for connectivity to the at least two smart devices embedded within the at least two smart drill bits.

The preceding description, therefore, is not meant to limit the scope of the disclosure but to provide sufficient disclosure to allow one of ordinary skill in the art to practice the disclosure without undue burden. It is further understood that the scope of the present disclosure fully encompasses other embodiments that may become obvious to those skilled in the art, and that the scope of the present disclosure is accordingly limited by nothing other than the appended claims.

It is understood that the preceding is merely a detailed description of some examples and embodiments of the present disclosure, and that numerous changes to the disclosed embodiments may be made in accordance with the disclosure made herein without departing from the spirit or scope of the disclosure. All references, including publications, patent applications and patents cited herein, are hereby incorporated by reference to the same extent as if each reference were individually and specifically indicated to be incorporated by reference and were set forth in its entirety herein.

Various features of the example embodiments described herein may be implemented using hardware, software or a combination thereof and may be implemented in one or more computer systems or other processing systems. However, the manipulations performed in these embodiments were often referred to in terms, such as determining, which are commonly associated with mental operations performed by a human operator.

No such capability of a human operator is necessary in any of the operations described herein. Rather, the operations may be completely implemented with machine operations. Useful machines for performing the operation of the exemplary embodiments presented herein include general purpose digital computers or similar devices.

As per the hardware, a CPU typically includes one or more components, such as one or more microprocessors for performing the arithmetic and/or logical operations required for program execution, and storage media, such as one or more disk drives or memory cards (e.g., flash memory) for program and data storage, and a random access memory for temporary data and program instruction storage. With respect to software, a CPU typically includes software resident on a storage media (e.g., a disk drive or memory card), which, when executed, directs the CPU in performing transmission and reception functions.

The CPU software may run on an operating system stored on the storage media, such as, for example, UNIX or Windows (e.g., NT, XP, Vista), Linux, and the like, and can adhere to various protocols such as the Ethernet, ATM, TCP/IP, CAN, LIN protocols and/or other connection or connectionless protocols. As is known in the art, CPUs can run different operating systems, and can contain different types of software, each type devoted to a different function, such as handling and managing data/information from a particular source, or transforming data/information from one format into another format. It should thus be clear that the embodiments described herein are not to be construed as being limited for use with any particular type of server computer, and that any other suitable type of device for facilitating the exchange and storage of information may be employed instead.

A CPU may be a single CPU, or may include multiple separate CPUs, wherein each is dedicated to a separate application, such as, for example, a data application, a voice application and a video application. Software embodiments of the example embodiments presented herein may be provided as a computer program product, or software, that may include an article of manufacture on a machine-accessible or non-transitory computer-readable medium (i.e., also referred to as “machine readable medium”) having instructions. The instructions on the machine-accessible or machine-readable medium may be used to program a computer system or other electronic device.

The machine-readable medium may include, but is not limited to, floppy diskettes, optical disks, CD-ROMs, magneto-optical disks, USB thumb drives and SD cards or other types of media/machine-readable medium suitable for storing or transmitting electronic instructions. The techniques described herein are not limited to any particular software configuration. They may find applicability in any computing or processing environment. The terms “machine-accessible medium,” “machine-readable medium” and “computer-readable medium” used herein shall include any non-transitory medium that is capable of storing, encoding or transmitting a sequence of instructions for execution by the machine (e.g., a CPU or other type of processing device) and that cause the machine to perform any one of the methods described herein. It is to be noted that it is common, as a person skilled in the art can contemplate, in the art to speak of software, in one form or another (e.g., program, procedure, process, application, module, unit, logic, and so on) as taking an action or causing a result. Such expressions are merely a shorthand way of stating that the execution of the software by a processing system causes the processor to perform an action to produce a result.

The use of the terms “a,” “an” and “the” and similar referents in the context of describing the invention (especially in the context of the following claims) is to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. It is understood that the preceding is merely a detailed description of some examples and embodiments of the present disclosure, and that numerous changes to the disclosed embodiments may be made in accordance with the disclosure made herein without departing from the spirit or scope of the disclosure. The preceding description, therefore, is not meant to limit the scope of the disclosure, but to provide sufficient disclosure to allow one of ordinary skill in the art to practice the disclosure without undue burden.

It is further understood that the scope of the present disclosure fully encompasses other embodiments that may become obvious to those skilled in the art. Features illustrated or described as part of one embodiment can be used in another embodiment to yield a still further embodiment. Thus, it is intended that the present disclosure cover such modifications and variations as come within the scope of the appended claims and their equivalents. It is to be understood by one of ordinary skill in the art that the present discussion is a description of exemplary embodiments only, and is not intended as limiting the broader aspects of the present disclosure, which broader aspects are embodied in the exemplary constructions.

As noted above, the apparatus, methods and systems described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The terms “invention,” “the invention,” “this invention,” “the present invention,” “disclosure,” “the disclosure” and “the present disclosure” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary.

This specification is a high-level overview of various aspects of the invention and introduces some of the concepts that are further described in the Detailed Description section below. This specification is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim. Although the present disclosure and its advantages have been described in detail, it should be understood that various changes, substitutions and alterations can be made herein without departing from the spirit and scope of the disclosure as defined in the appended claims.

1. A system comprising:
at least one drill bit having a first end and a second end located at a distal end of the first end;
a threaded portion in proximity to the second end;
a shaft connected to the threaded portion;
wherein the at least one drill bit is configured with at least one embedded processor configured to execute computer executable instructions, wherein the system is further configured to:
to provide real-time intra-operative reading of at least one variable measurement parameter of at least one bone during a certain medical procedure.

2. The system of claim 1, wherein the at least one variable measurement parameter comprises a length.

3. The system of claim 1, wherein the at least one variable measurement parameter comprises an alignment.

4. The system of claim 1, the at least one variable measurement parameter comprises a rotation.

5. The system of claim 1, wherein the at least one bone is a long bone.

6. The system of claim 1, wherein the certain medical procedure is a procedure relate to restoration of a fractured bone.

7. The system of claim 1, wherein the processor is further configured to connect with at least one sensor in order to sense a three-dimensional position in space.

8. The system of claim 1, wherein the processor is further configured to connect and communicate with at least one external device over at least one network.

9. A method of using a smart drill, the method comprising the steps of:
configuring at least one drill bit having a first end and a second end located at a distal end of the first end, a threaded portion in proximity to the second end, and a shaft connected to the threaded portion;
further configuring the at least one drill bit with at least one embedded processor in order to execute computer executable instructions; and
providing real-time intra-operative reading of at least one variable measurement parameter of at least one bone during a certain medical procedure.

10. The method of claim 9, wherein the at least one variable measurement parameter comprises a length.

11. The method of claim 9, wherein the at least one variable measurement parameter comprises an alignment.

12. The method of claim 9, wherein the at least one variable measurement parameter comprises a rotation.

13. The method of claim 9, wherein the at least one bone is a long bone.

14. The method of claim 9, wherein the certain medical procedure is a procedure related to restoration of a fractured bone.

15. The method of claim 9, wherein the processor is further configured to connect with at least one sensor in order to sense a three-dimensional position in space.

16. The method of claim 9, wherein the processor is further configured to connect and communicate with at least one external device over at least one network.

17. A smart drill comprising:
a first end and a second end located at a distal end of the first end;
a threaded portion in proximity to the second end;
a shaft connected to the threaded portion;
wherein the smart drill is configured with at least one embedded processor configured to execute computer executable instructions and configured to provide real-time intra-operative reading of at least one variable measurement parameter of at least one bone during a certain medical procedure.

18. The smart drill of claim 17, wherein the at least one variable measurement parameter comprises a length.

19. The smart drill of claim 17, wherein the at least one variable measurement parameter comprises an alignment.

20. The smart drill of claim 17, wherein the at least one variable measurement parameter comprises a rotation.
